(19) United States
(12) Patent Application Publication
(10) Pub. No.: US 2026/0207586 A1
(43) Pub. Date: Jul. 23, 2026

(54) PHARMACEUTICAL COMPOSITION COMPRISING LURBINECTEDIN, PREPARATION METHOD THEREFOR, AND USE THEREOF

(71) Applicant: BrightGene Bio-Medical Technology Co., Ltd., Suzhou, Jiangsu (CN)

(72) Inventors: Jiahan Li, Suzhou, Jiangsu (CN); Huifang Li, Suzhou, Jiangsu (CN); Yue Dong, Suzhou, Jiangsu (CN); Yangqing Huang, Suzhou, Jiangsu (CN)

(73) Assignee: BrightGene Bio-Medical Technology Co., Ltd., Suzhou, Jiangsu (CN)

(21) Appl. No.: 19/140,454

(22) PCT Filed: Dec. 21, 2023
(86) PCT No.: PCT/CN2023/140627

§ 371 Date: Jun. 18, 2025

(30) Foreign Application Priority Data
Dec. 22, 2022 (CN) 202211667008.4

(51) Int. Cl. A61K 31/4995; A61K 9/16; A61K 9/19

(52) U.S. Cl. CPC A61K 31/4995; A61K 9/1611; A61K 9/1623; A61K 9/19

(57) ABSTRACT

A pharmaceutical composition comprising lurbinectedin, a preparation method therefor, and use thereof. The pharmaceutical composition comprising lurbinectedin is a freeze-dried composition comprising lurbinectedin, a buffer derived from an inorganic acid, and a proppant. Lurbinectedin, the active ingredient of the pharmaceutical composition, can be completely dissolved and can ensure that the pH value of the pharmaceutical composition is stable during storage without influencing the quality and stability of the active pharmaceutical ingredient, featuring a low impurity level and long-term preservation potential, and thereby demonstrating significant application prospects in treating cancer and associated diseases. The preparation method for the pharmaceutical composition is simple and convenient to operate and cost-effective for production, facilitating large-scale production and contributing to the widespread use of pharmaceutical formulations.

The present disclosure claims priority of the Chinese Patent Application No. 202211667008.4, entitled “PHARMACEUTICAL COMPOSITION COMPRISING LURBINECTEDIN, PREPARATION METHOD THEREFOR, AND USE THEREOF”, filed with the China National Intellectual Property Administration on Dec. 22, 2022, which is incorporated herein by reference in its entirety.

### TECHNICAL FIELD

The present disclosure relates to the field of pharmaceuticals, and particularly to a pharmaceutical composition comprising lurbinectedin, a method for preparing same, and use thereof.

### BACKGROUND

Lung cancer is a disease in which malignant (cancerous) cells develop within the lung tissue. The two major types of lung cancer are small cell lung cancer (SCLC) and non-small cell lung cancer (NSCLC). Upon diagnosis, SCLC accounts for approximately 13%-15% of lung cancers. However, SCLC is a more invasive type of lung cancer, in which cancer cells tend to grow rapidly and metastasize more easily to other parts of the body. The most common treatment today involves treatment with cisplatin or carboplatin and etoposide. However, patients are susceptible to resistance to chemotherapy and recurrence.

Lurbinectedin (CAS No. 497871-47-3) is a derivative of compound ET-736 isolated from Ecteinacidia turbinata, in which a hydrogen atom in ET-736 is substituted with a methoxy group. The structure formula is shown below. In addition to its direct effect on cancer cells, lurbinectedin also inhibits the transcription and production of certain cytokines in tumor-associated macrophages that are essential for tumor growth. Lurbinectedin is suitable for the treatment of SCLC in adult patients with progression during or after platinum-based chemotherapy.

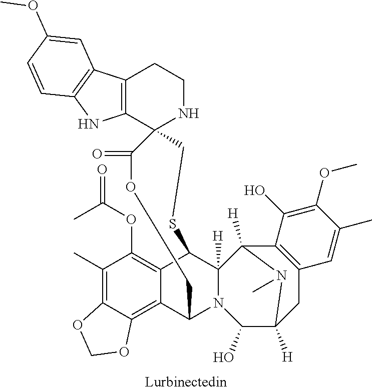

However, lurbinectedin is a complex compound with limited solubility in water and poor stability. It may be rapidly degraded at room temperature (25° C.), and can be stored for only 1 month at 5° C. under refrigeration. Therefore, lurbinectedin can only be stored at −20° C. for a long period, which greatly limits the clinical application of lurbinectedin. Therefore, it is very necessary to develop a lurbinectedin formulation that has good stability and stable efficacy and can be stored for a long time.

### SUMMARY

A first object of the present disclosure is to provide a pharmaceutical composition comprising lurbinectedin.

A second object of the present disclosure is to provide a method for preparing the pharmaceutical composition comprising lurbinectedin.

A third object of the present disclosure is to provide use of a pharmaceutical composition comprising lurbinectedin in the preparation of a drug for treating tumors.

For the above objects, the present disclosure provides the following embodiments: In a first aspect, the present disclosure provides a pharmaceutical composition comprising lurbinectedin, wherein the pharmaceutical composition is a lyophilized composition comprising: lurbinectedin, a buffer derived from an inorganic acid, and a bulking agent.

The buffer derived from the inorganic acid is selected from a combination of the inorganic acid and a salt of the inorganic acid or a combination of the inorganic acid and a base.

Furthermore, the buffer derived from the inorganic acid is selected from a combination of phosphoric acid and phosphate, a combination of hydrochloric acid and the base, a combination of sulfuric acid and the base, and a combination of phosphoric acid and the base, or selected from a combination of phosphoric acid and a phosphate salt, a combination of hydrochloric acid and the base, and a combination of sulfuric acid and the base, wherein the base is any one of sodium carbonate, potassium carbonate, NH₄OH, lithium hydroxide, sodium hydroxide, potassium hydroxide, sodium bicarbonate, and potassium bicarbonate, wherein the phosphate salt comprises a hydrate of the phosphate salt.

Preferably, the buffer derived from the inorganic acid is selected from a combination of phosphoric acid and sodium dihydrogen phosphate, a combination of phosphoric acid and potassium dihydrogen phosphate, a combination of hydrochloric acid and sodium hydroxide, a combination of sulfuric acid and sodium hydroxide, and a combination of phosphoric acid and sodium hydroxide, or selected from a combination of phosphoric acid and sodium dihydrogen phosphate, a combination of phosphoric acid and potassium dihydrogen phosphate, a combination of hydrochloric acid and sodium hydroxide, and a combination of sulfuric acid and sodium hydroxide.

More preferably, the buffer derived from the inorganic acid is selected from a combination of phosphoric acid and potassium dihydrogen phosphate and a combination of phosphoric acid and sodium dihydrogen phosphate, or selected from a combination of phosphoric acid and potassium dihydrogen phosphate.

Furthermore, in the pharmaceutical composition, the bulking agent is a disaccharide selected from any one of sucrose, trehalose, and lactose, and preferably, the bulking agent is sucrose.

Furthermore, in the solution prior to lyophilization of the lyophilized composition, the concentration of lurbinectedin is 0.4-0.6 mg/mL, and preferably, the concentration of lurbinectedin is 0.5 mg/mL.

Furthermore, in the pharmaceutical composition, the molar ratio of lurbinectedin to the bulking agent is 1:440-1:480, preferably, the molar ratio of lurbinectedin to the bulking agent is 1:450-1:470, and more preferably, the molar ratio of lurbinectedin to the bulking agent is 1:455-1:465. More preferably, the molar ratio of lurbinectedin to the bulking agent is 1:458.58.

Preferably, in the pharmaceutical composition, the bulking agent is sucrose, the molar ratio of lurbinectedin to sucrose is 1:458.58, and the corresponding mass ratio is 1:200.

Furthermore, in the pharmaceutical composition, the pH of the solution prior to lyophilization of the lyophilized composition is 3 to 5, and preferably, the pH of the solution prior to lyophilization of the lyophilized composition is 3.5 to 4.5.

Furthermore, the pharmaceutical composition consists of lurbinectedin, phosphoric acid, potassium dihydrogen phosphate, sucrose, potassium hydroxide, and water; the concentration of the lurbinectedin is 0.4 to 0.6 mg/mL, and the pH of the solution prior to lyophilization of the lyophilized composition is 3 to 5.

Furthermore, each 80 mL of the pharmaceutical composition comprises the following components:

| Component | Amount | |
|---|---|---|
| Lurbinectedin | 40 | mg |
| Sucrose | 8 | g |
| Phosphoric acid | 33.71 | mg |
| Potassium dihydrogen phosphate | 544 | mg |

| | |
|---|---|
| Potassium hydroxide/phosphoric acid | Proper amount |
| Water for injection | The balance to 80 mL |

Furthermore, the pharmaceutical composition consists of s lurbinectedin, phosphoric acid, sodium dihydrogen phosphate, sucrose, and water; the concentration of lurbinectedin is 0.4 to 0.6 mg/mL, and the pH of the solution prior to lyophilization of the lyophilized composition is 3 to 5.

Furthermore, each 80 mL of the pharmaceutical composition comprises the following components:

| Component | Amount | |
|---|---|---|
| Lurbinectedin | 40 | mg |
| Sucrose | 8 | g |
| Phosphoric acid | 11.5 | mg |
| Sodium dihydrogen | 294 | mg |
| phosphate | | |

| | |
|---|---|
| Water for injection | The balance |
| | to 80 mL |

In a second aspect, the present disclosure provides a method for preparing a pharmaceutical composition comprising lurbinectedin: mixing lurbinectedin, a buffer, a bulking agent, and water to form a mixture; and adjusting the pH of the mixture to obtain the pharmaceutical composition.

Furthermore, the method further comprises a lyophilization process after adjusting the pH.

The lyophilization process comprises three stages: a pre-freezing, a primary drying, and a secondary drying. For the pre-freezing, the temperature is −50 to 5° C.; for the primary drying, the temperature is −40 to −10° C., wherein the vacuum pressure is maintained at 0.05 to 0.5 mbar; for the secondary drying, the temperature is −25 to 45° C., wherein the vacuum pressure is maintained at 0.01 to 0.5 mbar.

Preferably, for the secondary drying, the vacuum pressure is maintained at 0.05 to 0.5 mbar.

Furthermore, the time for the pre-freezing is 0.5 to 8 h, the time for the primary drying (first drying) is 50 to 120 h, and the time for the secondary drying (second drying) is 24 to 65 h.

Furthermore, the method for preparing the pharmaceutical composition may be selected from any one of the following three preparation methods:

### Preparation Method I:

1) mixing lurbinectedin with an inorganic acid;
2) adding water, a base/a salt of the inorganic acid; and
3) adding a bulking agent, and adjusting the pH to obtain the pharmaceutical composition;

### Preparation Method II:

1) mixing lurbinectedin with an inorganic acid;
2) mixing a base/a salt of the inorganic acid, a bulking agent, and water, and adjusting the pH; and
3) mixing the solution obtained in step 1) and the solution obtained in step 2), and adjusting the pH to obtain the pharmaceutical composition; or

### Preparation Method III:

1) mixing an inorganic acid, a salt of the inorganic acid, and lurbinectedin;
2) mixing a bulking agent with water, and adjusting the pH; and
3) mixing the solution obtained in step 1) and the solution obtained in step 2), and adjusting the pH to obtain the pharmaceutical composition.

Furthermore, the method further comprises a lyophilization process after adjusting the pH, wherein the lyophilization process comprises three stages: a pre-freezing, a primary drying, and a secondary drying; for the pre-freezing, the temperature is −50 to 5° C.; for the primary drying, the temperature is −40 to −10° C., wherein the vacuum pressure is maintained at 0.1 to 0.5 mbar; for the secondary drying, the temperature is −25 to 25° C., wherein the vacuum pressure is maintained at 0.01 to 0.5 mbar.

Preferably, for the secondary drying, the vacuum pressure is maintained at 0.05 to 0.5 mbar.

Furthermore, the time for the pre-freezing is 0.5 to 8 h, the time for the primary drying (first drying) is 35 to 50 h, and the time for the secondary drying (second drying) is 28 h.

Preferably, for the pre-freezing, the temperature is −40 to 5° C.; for the primary drying, the temperature is −40 to −25° C.

Furthermore, the method further comprises a lyophilization process after adjusting the pH, wherein the lyophilization process comprises three stages: a pre-freezing, a primary drying, and a secondary drying; for the pre-freezing, the temperature is −50 to 5° C.; for the primary drying, the temperature is −40 to −10° C., wherein the vacuum pressure is maintained at 0.05 to 0.5 mbar; for the secondary drying, the temperature is −25 to 45° C., wherein the vacuum pressure is maintained at 0.01 to 0.5 mbar.

Preferably, for the secondary drying, the vacuum pressure is maintained at 0.05 to 0.5 mbar.

Furthermore, the time for the pre-freezing is 0.5 to 8 h, the time for the primary drying (first drying) is 50 to 120 h, and the time for the secondary drying (second drying) is 24 to 65 h. Preferably, the time for the primary drying (first drying) is 50 to 100 h, and the time for the secondary drying (second drying) is 24 to 46 h.

In a third aspect, the present disclosure provides use of a pharmaceutical composition comprising lurbinectedin in the preparation of a drug for treating tumors.

In the pharmaceutical composition provided by the present disclosure, the proper selection of excipients such as the buffer and the bulking agent allows lurbinectedin, as the active ingredient, to keep good stability in the lyophilization and solves the problem of poor solubility of lurbinectedin in water. Therefore, a lurbinectedin pharmaceutical composition that meets the requirement of intravenous drip infusion and can be stably stored is obtained, and the pharmaceutical composition has good application prospects in the preparation of anti-tumor drugs.

By utilizing the combination of an inorganic acid and a salt of the inorganic acid or the combination of an inorganic acid and a base as the buffer, the present disclosure ensures the complete dissolution of the active ingredient lurbinectedin of the pharmaceutical composition in the preparation process and a stable pH of the pharmaceutical composition in the storage process, without affecting the quality and stability of the active ingredient of the pharmaceutical composition. The pharmaceutical composition possesses a low impurity content and can be stored for a long time, with an osmotic pressure meeting the requirement of intravenous drip medicament (>100 mL; 260 to 330 mOsmol/kg).

The method for preparing the pharmaceutical composition provided by the present disclosure features ease to operate and cost-efficiency, facilitates massive production, and may thus contribute to the broad application of the pharmaceutical formulation.

### DETAILED DESCRIPTION

Embodiments described herein are provided to illustrate the present disclosure, but the disclosure is not limited to these embodiments. The present disclosure is not limited to the configurations described below, and various modifications are possible within the claimed scope of the present disclosure, and embodiments and examples obtained by appropriately combining the technical means disclosed in the different embodiments and examples are also included in the technical scope of the present disclosure.

Unless otherwise defined, all technical and scientific terms used herein have the same meanings as those commonly understood by those of ordinary skill in the art to which the present disclosure belongs.

In the present disclosure, “/” refers to “and” or “or”.

In the specification, the numerical ranges indicated by the use of “numerical value A to numerical value B” or “numerical value A-numerical value B” refer to ranges including the endpoint numerical values A and B.

In the specification, the meaning of “may” includes both the meaning of performing a certain process and the meaning of not performing a certain process.

Unless otherwise stated, in the specification, procedures without specified conditions shall be conducted according to conventional conditions or conditions recommended by the manufacturers. The adopted reagents or instruments, without manufacturers, are all commercially available, conventional products.

All units used in the specification are international standard units, and the numerical values and numerical ranges appearing in the present disclosure should be understood to include unavoidable systematic errors in industrial production.

Reference in the specification to “some specific/preferred embodiments”, “some other specific/preferred embodiments”, “embodiments”, and the like, means that a particular element (e.g., feature, structure, property, and/or characteristic) described in relation with the embodiment is included in at least one embodiment described herein, and may or may not be present in other embodiments. In addition, it will be appreciated that the described elements may be combined in any proper manner in the various embodiments.

The concentrations of phosphoric acid, sulfuric acid, hydrochloric acid, and sodium hydroxide used in the examples of the present disclosure were all 0.1 mol/L. For example, when 0.1 mol/L phosphoric acid was used, if 3.44 mL of phosphoric acid was used in the formula, the theoretical number of moles was 0.344 mmol, and the weight was 0.344 mmol×97.99 g/mol=33.71 mg.

### Example 1: Formulation Example I

A lurbinectedin solution before lyophilization was prepared with lurbinectedin as the active ingredient, a combination of phosphoric acid/potassium dihydrogen phosphate as the buffer, phosphoric acid/potassium hydroxide as the pH adjusting agent, sucrose as the bulking agent, and water for injection as the solvent. The composition and the specific amounts of the components in Formulation Example I are shown in Table 1:

TABLE 1
Formulation Example I

| Component | Effect | Amount | |
|---|---|---|---|
| Lurbinectedin | Active ingredient | 40 | mg |
| Sucrose | Bulking agent | 8 | g |
| Phosphoric acid | Buffer | 33.71 | mg |
| Potassium dihydrogen | Buffer | 544 | mg |

| | | |
|---|---|---|
| phosphate | | |
| Potassium hydroxide/ | pH adjusting | Proper |
| phosphoric acid | agent | amount |
| Water for injection | Solvent | The balance |
| | | to 80 mL |

### Preparation:

1. 40 mg of lurbinectedin was added to 33.71 mg of phosphoric acid, and the mixture was stirred to completely dissolve the drug, so as to obtain a drug solution;
2. 70% of water for injection was added into the drug solution, the mixture was uniformly stirred, 544 mg of potassium dihydrogen phosphate was then added into the drug solution, and the mixture was stirred for dissolution;
3. 8 g of sucrose was added to the solution obtained in step 2, and the mixture was stirred to dissolve the sucrose and adjusted to pH 4.0±0.2 by using a phosphoric acid solution or a potassium hydroxide solution;
4. The formulated solution was adjusted to the total weight and filled into vials before the vials were half-stoppered.

The above solution was then lyophilized to obtain the lyophilized formulation, and the appearance, water content, and related substances were inspected. In this example, Lyophilization Process I of Example 12 was employed. The lyophilized formulation was tested for storage stability in an acceleration condition (25° C., 60% RH), and the results are shown in Table 2:

TABLE 2
The stability study of the lyophilized
formulation produced by Formulation
Example I

| | | Time | | | |
|---|---|---|---|---|---|
| | | | Accel- | Accel- | Accel- |
| | | | eration, | eration, | eration, |
| | | 0 days | 1M | 2M | 3M |
| Appearance | | White | White | White | White |
| | | solid mass | solid mass | solid mass | solid mass |
| Water content (%) | | 1.99 | 1.82 | 1.94 | 2.20 |
| Related | RRT0.16 | / | / | / | / |
| substances | RRT0.23 | / | / | / | / |
| (area | RRT0.41 | / | / | / | / |
| normalization, | RRT0.61 | / | / | / | / |
| % | RRT0.69 | / | 1 | / | / |
| | RRT0.84 | 0.11 | 0.15 | 0.16 | 0.16 |
| | RRT1.10 | / | / | 1 | / |
| | RRT1.28 | 0.06 | 0.05 | 0.06 | 0.06 |
| | RRT1.36 | 0.06 | 0.05 | 0.07 | 0.07 |
| | Maximum | 0.11 | 0.15 | 0.16 | 0.16 |
| | unknown | | | | |
| | individual | | | | |
| | impurity | | | | |
| | Total | 0.39 | 0.35 | 0.46 | 0.41 |
| | impurities | | | | |

Note: (1) ″/″ indicates not detected or less than 0.05%;

As can be seen from Table 2, by using the combination of phosphoric acid and potassium dihydrogen phosphate as the buffer, the active ingredient lurbinectedin was completely dissolved in the solution. The lyophilized formulation prepared by lyophilization was qualified in appearance, water content, and related substances, and exhibited good stability in the acceleration conditions and substantially no changes in total impurities after storage for 3 months in the conditions of 25° C. and 60% RH, indicating that the combination of potassium dihydrogen phosphate and phosphoric acid as the buffer of the lurbinectedin formulation can improve the stability of lurbinectedin in the storage process.

### Example 2: Formulation Example II

A lurbinectedin solution before lyophilization was prepared with lurbinectedin as the active ingredient, a combination of phosphoric acid/sodium dihydrogen phosphate as the buffer, phosphoric acid/sodium hydroxide as the pH adjusting agent, sucrose as the bulking agent, and water for injection as the solvent. The composition and the specific amounts of the components in Formulation Example II are shown in Table 3:

TABLE 3
Formulation Example II

| Component | Effect | Amount | |
|---|---|---|---|
| Lurbinectedin | Active ingredient | 40 | mg |
| Sucrose | Bulking agent | 8 | g |
| Phosphoric acid | Buffer | 33.71 | mg |
| Sodium dihydrogen | Buffer | 480 | mg |
| phosphate | | | |

| | | |
|---|---|---|
| Sodium hydroxide/ | pH adjusting | Proper amount |
| phosphoric acid | agent | |
| Water for injection | Solvent | The balance |
| | | to 80 mL |

### Preparation:

1. 40 mg of lurbinectedin was added to 33.71 mg of phosphoric acid, and the mixture was stirred to completely dissolve the drug, so as to obtain a drug solution;
2. 70% of water for injection was added into the drug solution, the mixture was uniformly stirred, 480 mg of sodium dihydrogen phosphate was then added into the drug solution, and the mixture was stirred for dissolution;
3. 8 g of sucrose was added to the solution obtained in step 2, and the mixture was stirred to dissolve the sucrose and adjusted to pH 4.0±0.2 by using a phosphoric acid solution or a sodium hydroxide solution;
4. The formulated solution was adjusted to the total weight and filled into vials before the vials were half-stoppered.

The above solution was then lyophilized to obtain the lyophilized formulation, and the appearance, water content, and related substances were inspected. In this example, Lyophilization Process I of Example 12 was employed. The lyophilized formulation was tested for storage stability in an acceleration condition (25° C., 60% RH), and the results are shown in Table 4:

TABLE 4
The stability study of the lyophilized formulation produced by Formulation
Example II

| | | Time | | | |
|---|---|---|---|---|---|
| | | | Accel- | Accel- | Accel- |
| | | | eration, | eration, | eration, |
| | | 0 days | 1M | 2M | 3M |
| Appearance | | White | White | White | White |
| | | solid mass | solid mass | solid mass | solid mass |
| Water content (%) | | 2.01 | 1.98 | 2.10 | 2.21 |
| Related | RRT0.16 | / | / | / | / |
| substances | RRT0.23 | / | / | / | / |
| (area | RRT0.41 | / | / | / | / |
| normal- | RRT0.61 | / | / | / | / |
| ization, %) | RRT0.69 | / | / | / | / |
| | RRT0.84 | 0.12 | 0.17 | 0.17 | 0.17 |
| | RRT1.10 | / | / | / | / |
| | RRT1.28 | 0.05 | 0.06 | 0.05 | 0.06 |
| | RRT1.36 | 0.06 | 0.06 | 0.07 | 0.08 |
| | Maximum | 0.10 | 0.16 | 0.17 | 0.16 |
| | unknown | | | | |
| | individual | | | | |
| | impurity | | | | |
| | Total | 0.41 | 0.40 | 0.45 | 0.47 |
| | impurities | | | | |

Note: (1) ″/″ indicates not detected or less than 0.05%;

As can be seen from Table 4, by using the combination of sodium dihydrogen phosphate and phosphoric acid as the buffer, the active ingredient lurbinectedin was completely dissolved in the solution. The lyophilized formulation prepared by lyophilization was qualified in appearance, water content, and related substances, and exhibited good stability in the acceleration conditions and an insignificant change in total impurities from 0.41 to 0.47 after storage for 3 months in the conditions of 25° C. and 60% RH, indicating that the combination of sodium dihydrogen phosphate and phosphoric acid as the buffer of the lurbinectedin formulation can improve the stability of lurbinectedin in the storage process.

### Example 3: Formulation Example III

A lurbinectedin solution before lyophilization was prepared with lurbinectedin as the active ingredient, a combination of hydrochloric acid/sodium hydroxide as the buffer, hydrochloric acid/sodium hydroxide as the pH adjusting agent, sucrose as the bulking agent, and water for injection as the solvent. The composition and the specific amounts of the components in Formulation Example III are shown in Table 5:

TABLE 5
Formulation Example III

| Component | Effect | Amount | |
|---|---|---|---|
| Lurbinectedin | Active ingredient | 40 | mg |
| Sucrose | Bulking agent | 8 | g |
| Hydrochloric acid | Buffer | 12.54 | mg |

| | | |
|---|---|---|
| | | (based on |
| | | hydrogen chloride) |
| Sodium hydroxide | Buffer | Proper amount |
| Sodium hydroxide/ | pH adjusting | Proper amount |
| hydrochloric acid | agent | |
| Water for injection | Solvent | The balance |
| | | to 80 mL |

### Preparation:

1. 40 mg of lurbinectedin was added to 12.54 mg of hydrochloric acid, and the mixture was stirred to completely dissolve the drug, so as to obtain a drug solution;
2. 70% of water for injection was added into the drug solution, and the mixture was uniformly stirred;
3. 8 g of sucrose was added to the solution obtained in step 2, and the mixture was stirred to dissolve the sucrose and adjusted to pH 4.0±0.2 by using a hydrochloric acid solution or a sodium hydroxide solution;
4. The formulated solution was adjusted to the total weight and filled into vials before the vials were half-stoppered.

The above solution was then lyophilized to obtain the lyophilized lurbinectedin formulation. The appearance, water content, and related substances were inspected. In this example, Lyophilization Process I of Example 12 was employed. The lyophilized formulation was tested for storage stability in an acceleration condition (25° C., 60% RH), and the results are shown in Table 6:

TABLE 6
The stability study of the lyophilized formulation produced by Formulation
Example III

| | | Time | | | |
|---|---|---|---|---|---|
| | | | Accel- | Accel- | Accel- |
| | | | eration, | eration, | eration, |
| | | 0 days | 1M | 2M | 3M |
| Appearance | | White | White | White | White |
| | | solid mass | solid mass | solid mass | solid mass |
| Water content (%) | | 2.12 | 2.23 | 2.26 | 2.34 |
| Related | RRT0.16 | / | / | / | / |
| substances | RRT0.23 | / | / | / | / |
| (area | RRT0.41 | / | / | / | / |
| normalizat | RRT0.61 | | / | / | / |
| ion, %) | RRT0.69 | / | / | / | / |
| | RRT0.84 | 0.15 | 0.17 | 0.18 | 0.21 |
| | RRT1.10 | / | / | / | / |
| | RRT1.28 | 0.06 | 0.08 | 0.07 | 0.07 |
| | RRT1.36 | 0.08 | 0.09 | 0.09 | 0.09 |
| | Maximum | 0.17 | 0.21 | 0.23 | 0.26 |
| | unknown | | | | |
| | individual | | | | |
| | impurity | | | | |
| | Total | 0.48 | 0.57 | 0.62 | 0.69 |
| | impurities | | | | |

Note: (1) ″/″ indicates not detected or less than 0.05%;

As can be seen from Table 6, by using the combination of hydrochloric acid and sodium hydroxide as the buffer, the active ingredient lurbinectedin was completely dissolved in the solution. The lyophilized formulation prepared by lyophilization was qualified in appearance, water content, and related substances, and exhibited good stability in the acceleration conditions and a mild change in total impurities from 0.48 to 0.69 after storage for 3 months in the conditions of 25° C. and 60% RH, indicating that the combination of hydrochloric acid and sodium hydroxide as the buffer of the lurbinectedin formulation can ensure the stability of lurbinectedin in the storage process.

### Example 4: Formulation Example IV

A lurbinectedin solution before lyophilization was prepared with lurbinectedin as the active ingredient, a combination of sulfuric acid/sodium hydroxide as the buffer, sulfuric acid/sodium hydroxide as the pH adjusting agent, sucrose as the bulking agent, and water for injection as the solvent. The composition and the specific amounts of the components in Formulation Example IV are shown in Table 7:

TABLE 7
Formulation Example IV

| Component | Effect | Amount | |
|---|---|---|---|
| Lurbinectedin | Active ingredient | 40 | mg |
| Sucrose | Bulking agent | 8 | g |
| Sulfuric acid | Buffer | 33.71 | mg |

| | | |
|---|---|---|
| Sodium hydroxide | Buffer | Proper amount |
| Sodium hydroxide/ | pH adjusting | Proper amount |
| sulfuric acid | agent | |
| Water for injection | Solvent | The balance |
| | | to 80 mL |

### Preparation:

1. 40 mg of lurbinectedin was added to 33.71 mg of sulfuric acid, and the mixture was stirred to completely dissolve the drug, so as to obtain a drug solution;
2. 70% of water for injection was added into the drug solution, and the mixture was uniformly stirred;
3. 8 g of sucrose was added to the solution obtained in step 2, and the mixture was stirred to dissolve the sucrose and adjusted to pH 4.0±0.2 by using a sulfuric acid solution or a sodium hydroxide solution;
4. The formulated solution was adjusted to the total weight and filled into vials before the vials were half-stoppered.

The above solution was then lyophilized to obtain the lyophilized lurbinectedin formulation, and the appearance, water content, and related substances were inspected. In this example, Lyophilization Process I of Example 12 was employed. The lyophilized formulation was tested for storage stability in an acceleration condition (25° C., 60% RH), and the results are shown in Table 8:

TABLE 8
The stability study of the lyophilized formulation produced by Formulation
Example IV

| | | Time | | | |
|---|---|---|---|---|---|
| | | | Acceleration, | Acceleration, | Acceleration, |
| | | 0 days | 1M | 2M | 3M |
| Appearance | | White | White | White | White |
| | | solid mass | solid mass | solid mass | solid mass |
| Water content (%) | | 2.17 | 2.09 | 2.29 | 2.40 |
| Related | RRT0.16 | / | / | / | / |
| substances | RRT0.23 | / | / | / | / |
| (area | RRT0.41 | / | | / | / |
| normalization, | RRT0.61 | / | / | / | 1 |
| | RRT0.69 | / | / | / | / |
| | RRT0.84 | 0.24 | 0.29 | 0.32 | 0.37 |
| | RRT1.10 | / | / | / | / |
| | RRT1.28 | 0.06 | 0.07 | 0.06 | 0.08 |
| | RRT1.36 | 0.07 | 0.08 | 0.08 | 0.09 |
| | Maximum | 0.23 | 0.27 | 0.29 | 0.33 |
| | unknown | | | | |
| | individual impurity | | | | |
| | Total impurities | 0.67 | 0.79 | 0.84 | 0.89 |

Note: (1) ″/″ indicates not detected or less than 0.05%.

As can be seen from Table 8, by using the combination of sulfuric acid and sodium hydroxide as the buffer, the active ingredient lurbinectedin was completely dissolved in the solution. The lyophilized formulation prepared by lyophilization was qualified in appearance, water content, and related substances, and exhibited good stability in the acceleration conditions and a mild change in total impurities from 0.67 to 0.89 after storage for 3 months in the conditions of 25° C. and 60% RH, indicating that the combination of sulfuric acid and sodium hydroxide as the buffer of the lurbinectedin formulation can ensure the stability of lurbinectedin in the storage process.

### Example 5: Formulation Example V

A lurbinectedin solution before lyophilization was prepared with lurbinectedin as the active ingredient, a combination of phosphoric acid/sodium dihydrogen phosphate as the buffer, sucrose as the bulking agent, and water for injection as the solvent. The composition and the specific amounts of the components in Formulation Example V are shown in Table 9:

TABLE 9
Formulation Example V

| Component | Effect | Amount | |
|---|---|---|---|
| Lurbinectedin | Active ingredient | 40 | mg |
| Sucrose | Bulking agent | 8 | g |
| Phosphoric acid | Buffer | 11.5 | mg |
| Sodium dihydrogen | Buffer | 294 | mg |

| | | |
|---|---|---|
| phosphate | | |
| Water for | Solvent | The balance |
| injection | | to 80 mL |

### Preparation:

1. 40 mg of lurbinectedin was added to 11.5 mg of phosphoric acid, and the mixture was stirred to completely dissolve the drug, so as to obtain a drug solution;
2. 8 g of sucrose and 294 mg of sodium dihydrogen phosphate were added to 70% of water for injection, and the mixture was stirred for dissolving;
3. The solution obtained in step 1 was completely added to the solution obtained in step 2, and the mixture was uniformly stirred to obtain a solution of pH 4.0±0.2;
4. The formulated solution was adjusted to the total weight and filled into vials before the vials were half-stoppered.

The above solution was then lyophilized to obtain the lyophilized lurbinectedin formulation.

In this example, the lyophilization procedures are as follows:

| | Temperature | Set time | Duration | Vacuum |
|---|---|---|---|---|
| Procedure | (°C) | (min) | (h) | mbar |
| Pre- | -5 ° C. | 10 | 1 | / |
| freezing | -40 ° C. | 30 | 5 | / |
| Primary | -25 ° C. | 60 | 50 | 0.1 |
| drying | | | | |
| Secondary | 25 ° C. | 330 | 20 | 0.03 |
| drying | 25 ° C. | 1 | 4 | 0.00 |

### Example 6: Formulation Example VI

A lurbinectedin solution before lyophilization was prepared with lurbinectedin as the active ingredient, a combination of phosphoric acid/sodium dihydrogen phosphate as the buffer, sodium hydroxide as the pH adjusting agent, sucrose as the bulking agent, and water for injection as the solvent. The composition and the specific amounts of the components in Formulation Example VI are shown in Table 10:

TABLE 10
Formulation Example VI

| Component | Effect | Amount | |
|---|---|---|---|
| Lurbinectedin | Active ingredient | 40 | mg |
| Sucrose | Bulking agent | 8 | g |
| Phosphoric acid | Buffer | 33.71 | mg |
| Sodium dihydrogen | Buffer | 480 | mg |

| | | |
|---|---|---|
| phosphate | | |
| Sodium hydroxide | pH adjusting | Proper |
| | agent | amount |
| Water for injection | Solvent | The balance |
| | | to 80 mL |

### Preparation:

1. 40 mg of lurbinectedin was added to 33.71 mg of phosphoric acid, and the mixture was stirred to completely dissolve the drug, so as to obtain a drug solution;
2. 8 g of sucrose and 480 mg of sodium dihydrogen phosphate were added to 70% of water for injection, and the mixture was stirred for dissolving;
3. The solution obtained in step 1 was completely added to the solution obtained in step 2, and the mixture was uniformly stirred and adjusted to pH 4.0±0.2 with a sodium hydroxide solution;
4. The formulated solution was adjusted to the total weight and filled into vials before the vials were half-stoppered.

The above solution was then lyophilized to obtain the lyophilized lurbinectedin formulation.

In this example, the lyophilization procedures are as follows:

| | Temperature | Set time | Duration | Vacuum |
|---|---|---|---|---|
| Procedure | (° C.) | (min) | (h) | mbar |
| Pre-freezing | -5° C. | 10 | 1 | / |
| | -40° C. | 30 | 5 | / |
| Primary drying | -25° C. | 60 | 50 | 0.1 |
| Secondary | 25° C. | 330 | 20 | 0.03 |
| drying | | | | |
| | 25° C. | 1 | 4 | 0.00 |

### Example 7: Formulation Example VII

A lurbinectedin solution before lyophilization was prepared with lurbinectedin as the active ingredient, a combination of phosphoric acid/potassium dihydrogen phosphate as the buffer, sodium hydroxide as the pH adjusting agent, sucrose as the bulking agent, and water for injection as the solvent. The composition and the specific amounts of the components in Formulation Example VII are shown in Table 11:

TABLE 11
Formulation Example VII

| Component | Effect | Amount | |
|---|---|---|---|
| Lurbinectedin | Active ingredient | 40 | mg |
| Sucrose | Bulking agent | 8 | g |
| Phosphoric acid | Buffer | 23.71 | mg |
| Potassium dihydrogen | Buffer | 544 | mg |

| | | |
|---|---|---|
| phosphate | | |
| Sodium hydroxide | pH adjusting | Proper |
| | agent | amount |
| Water for injection | Solvent | The balance |
| | | to 80 mL |

### Preparation:

1. 40 mg of lurbinectedin was added to 23.71 mg of phosphoric acid, and the mixture was stirred to completely dissolve the drug, so as to obtain a drug solution;
2. 8 g of sucrose and 544 mg of potassium dihydrogen phosphate were added to 70% of water for injection, and the mixture was stirred for dissolving;
3. The solution obtained in step 1 was completely added to the solution obtained in step 2, and the mixture was uniformly stirred and adjusted to pH 4.0±0.2 with a sodium hydroxide solution;
4. The formulated solution was adjusted to the total weight and filled into vials before the vials were half-stoppered.

The above solution was then lyophilized to obtain the lyophilized formulation.

In this example, the lyophilization procedures are as follows:

| | Temperature | Set time | Duration | Vacuum |
|---|---|---|---|---|
| Procedure | (° C.) | (min) | (h) | (mbar) |
| Pre- | -5° C. | 10 | 1 | / |
| freezing | -40° C. | 30 | 5 | / |
| Primary | -25° C. | 60 | 50 | 0.1 |
| drying | | | | |
| Secondary | 25° C. | 330 | 20 | 0.03 |
| drying | 25° C. | 1 | 4 | 0.00 |

### Example 8: Formulation Example VIII

A lurbinectedin solution before lyophilization was prepared with lurbinectedin as the active ingredient, a combination of phosphoric acid/sodium hydroxide as the buffer, sucrose as the bulking agent, phosphoric acid as the pH adjusting agent, and water for injection as the solvent. The composition and the specific amounts of the components in Formulation Example VIII are shown in Table 12:

TABLE 12
Formulation Example VIII

| Component | Effect | Amount |
|---|---|---|
| Lurbinectedin | Active ingredient | 40 mg |
| Sucrose | Bulking agent | 8 g |
| Phosphoric acid | Buffer | 127.1 mg |
| Sodium hydroxide | Buffer | 51 mg |
| Phosphoric acid | pH adjusting agent | Proper amount |
| Water for injection | Solvent | The balance to 80 mL |

### Preparation:

1. 40 mg of lurbinectedin was added to 127.1 mg of phosphoric acid, and the mixture was stirred to completely dissolve the drug, so as to obtain a drug solution;
2. 8 g of sucrose and 51 mg of sodium hydroxide were added to 70% of water for injection, and the mixture was stirred for dissolving;
3. The solution obtained in step 1 was completely added to the solution obtained in step 2, and the mixture was uniformly stirred and adjusted to pH 4.0±0.2 with phosphoric acid;
4. The formulated solution was adjusted to the total weight and filled into vials before the vials were half-stoppered.

The above solution was further lyophilized to obtain the lyophilized composition.

In this example, the lyophilization procedures are as follows:

| | Temperature | Set time | Duration | Vacuum |
|---|---|---|---|---|
| Procedure | (° C.) | min | (h) | (mbar) |
| Pre-freezing | −5° C. | 10 | 1 | / |
| | −40° C. | 30 | 5 | / |
| Primary drying | −25° C. | 60 | 50 | 0.1 |
| Secondary drying | 25° C. | 330 | 20 | 0.03 |
| | 25° C. | 1 | 4 | 0.00 |

### Example 9: Formulation Example IX

A lurbinectedin solution before lyophilization was prepared with lurbinectedin as the active ingredient, a combination of phosphoric acid/sodium hydroxide as the buffer, sucrose as the bulking agent, and water for injection as the solvent. The composition and the specific amounts of the components in Formulation Example IX are shown in Table 13:

TABLE 13
Formulation Example IX

| Component | Effect | Amount |
|---|---|---|
| Lurbinectedin | Active ingredient | 40 mg |
| Sucrose | Bulking agent | 8 g |
| Phosphoric acid | Buffer | 139.1 mg |
| Sodium hydroxide | Buffer | 51 mg |
| Water for injection | Solvent | The balance to 80 mL |

### Preparation:

1. 40 mg of lurbinectedin was added to 139.1 mg of phosphoric acid, and the mixture was stirred to completely dissolve the drug, so as to obtain a drug solution;
2. 8 g of sucrose and 51 mg of sodium hydroxide were added to 70% of water for injection, and the mixture was stirred for dissolving;
3. The solution obtained in step 1 was completely added to the solution obtained in step 2, and the mixture was uniformly stirred to obtain a solution of pH 4.0±0.2;
4. The formulated solution was adjusted to the total weight and filled into vials before the vials were half-stoppered.

The above solution was further lyophilized to obtain the lyophilized formulation.

In this example, the lyophilization procedures are as follows:

| | Temperature | Set time | Duration | Vacuum |
|---|---|---|---|---|
| Procedure | (° C) | min | (h) | mbar |
| Pre-freezing | −5° C. | 10 | 1 | / |
| | −40° C. | 30 | 5 | / |
| Primary drying | −25° C. | 60 | 50 | 0.1 |
| Secondary drying | 25° C. | 330 | 20 | 0.03 |
| | 25° C. | 1 | 4 | 0.00 |

### Comparative Example I

A lurbinectedin solution before lyophilization was prepared with lurbinectedin as the active ingredient, a combination of lactic acid/sodium hydroxide as the buffer, sucrose as the bulking agent, and water for injection as the solvent. The composition and the specific amounts of the components in this formulation are shown in Table 14:

TABLE 14
Comparative Example I Formulation

| Component | Effect | Amount |
|---|---|---|
| Lurbinectedin | Active ingredient | 40 mg |
| Sucrose | Bulking agent | 8 g |
| Lactic acid | Buffer | 221 mg |
| Sodium hydroxide | Buffer | 51 mg |
| Water for injection | Solvent | The balance to 80 mL |

### Preparation:

1. 40 mg of lurbinectedin was added to 45 mg of lactic acid, and the mixture was stirred to completely dissolve the drug, so as to obtain a drug solution;
2. 8 g of sucrose, 51 mg of sodium hydroxide, and 176 mg of lactic acid were added to 70% of water for injection, and the mixture was stirred for dissolving;
3. The solution obtained in step 1 was completely added to the solution obtained in step 2, and the mixture was uniformly stirred;
4. The formulated solution was adjusted to the total weight and filled into vials before the vials were half-stoppered.

The above solution was further lyophilized to obtain the lyophilized formulation.

In this example, the lyophilization procedures are as follows:

| | Temperature | Set time | Duration | Vacuum |
|---|---|---|---|---|
| Procedure | (° C.) | min | (h) | mbar |
| Pre-freezing | −5° C. | 10 | 1 | / |
| | −40° C. | 30 | 5 | / |
| Primary drying | −25° C. | 60 | 50 | 0.1 |
| Secondary drying | 25° C. | 330 | 20 | 0.03 |
| | 25° C. | 1 | 4 | 0.00 |

### Example 10: The Stability Study of the Lyophilized Formulation Produced by Formulation Example V to IX and Comparative Example I

The appearance, water content, and related substances of the lyophilized formulations obtained from formulas V to IX and Comparative Example I were inspected. The lyophilized formulations were tested for storage stability in acceleration conditions (25° C./60% RH, 40° C.), and the results are shown in Table 15:

TABLE 15
The stability study of the lyophilized formulation produced by
Formulation Example V to IX and Comparative Example I
Formulation Example V

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.1 | 4.0 | 4.1 | 4.0 |
| Water content (%) | | 1.27 | 2.39 | 1.74 | 2.53 |
| Related | Impurity A | 0.02 | 0.04 | 0.07 | 0.03 |
| substances | Impurity B | 0.01 | 0.01 | 0.01 | 0.01 |
| (%) | Impurity C | 0.02 | 0.02 | 0.03 | 0.03 |
| | Maximum | 0.04 | 0.06 | 0.07 | 0.04 |
| | unknown | | | | |
| | individual | | | | |
| | impurity | | | | |
| | Total | 0.29 | 0.30 | 0.35 | 0.23 |
| | impurities | | | | |

Formulation Example VI

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.2 | 4.2 | 4.2 | 4.1 |
| Water content (%) | | 2.74 | 2.80 | 2.83 | 2.58 |
| Related | Impurity A | 0.02 | 0.10 | 0.19 | 0.04 |
| substances | Impurity B | 0.01 | 0.01 | 0.01 | 0.01 |
| (%) | Impurity C | 0.02 | 0.02 | 0.02 | 0.03 |
| | Maximum | 0.04 | 0.06 | 0.10 | 0.04 |
| | unknown | | | | |
| | individual | | | | |
| | impurity | | | | |
| | Total | 0.20 | 0.39 | 0.56 | 0.25 |
| | impurities | | | | |

Formulation Example VII

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.2 | / | 4.1 | 4.1 |
| Water content (%) | | 2.25 | 2.15 | 2.41 | 1.92 |
| Related | Impurity A | 0.03 | 0.08 | 0.20 | 0.04 |
| substances | Impurity B | 0.02 | 0.01 | 0.01 | 0.01 |
| (%) | Impurity C | 0.02 | 0.02 | 0.02 | 0.02 |
| | Maximum | 0.04 | 0.04 | 0.12 | 0.04 |
| | unknown | | | | |
| | individual | | | | |
| | impurity | | | | |
| | Total | 0.22 | 0.36 | 0.60 | 0.24 |
| | impurities | | | | |

Formulation Example VIII

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.1 | 4.0 | 4.0 | 4.0 |
| Water content (%) | | 1.37 | 1.45 | 1.59 | 1.84 |
| Related | Impurity A | 0.02 | 0.05 | 0.07 | 0.03 |
| substances | Impurity B | 0.01 | 0.02 | 0.01 | 0.02 |
| (%) | Impurity C | 0.02 | 0.02 | 0.02 | 0.02 |
| | Maximum | 0.04 | 0.06 | 0.07 | 0.04 |
| | unknown | | | | |
| | individual | | | | |
| | impurity | | | | |
| | Total | 0.29 | 0.34 | 0.39 | 0.26 |
| | impurities | | | | |

Formulation Example IX

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.1 | 4.1 | 4.1 | 4.1 |
| Water content (%) | | 1.88 | 1.77 | 1.93 | 2.34 |
| Related | Impurity A | 0.02 | 0.05 | 0.13 | 0.03 |
| substances | Impurity B | 0.01 | 0.02 | 0.02 | 0.02 |
| (%) | Impurity C | 0.02 | 0.02 | 0.02 | 0.03 |
| | Maximum | 0.04 | 0.05 | 0.06 | 0.04 |
| | unknown | | | | |
| | individual | | | | |
| | impurity | | | | |
| | Total | 0.28 | 0.34 | 0.45 | 0.26 |
| | impurities | | | | |

Comparative Example I

| | | Condition | | | |
|---|---|---|---|---|---|
| | | | | 40° C., | 25° C., |
| | | 0D | 40° C., 10D | 1M | 1M |
| Appearance | | White | White | White | White |
| | | solid | solid | solid | solid |
| | | mass | mass | mass | mass |
| pH value | | 4.0 | 4.0 | 3.9 | 3.9 |
| Water content (%) | | 1.50 | 1.81 | 2.31 | 1.58 |
| Related | Impurity A | 0.02 | 0.05 | 0.08 | 0.02 |
| substances | Impurity B | 0.01 | 0.02 | 0.01 | 0.01 |
| (%) | Impurity C | 0.02 | 0.02 | 0.03 | 0.03 |
| | Maximum | 0.04 | 0.06 | 0.09 | 0.04 |
| | unknown | | | | |
| | individual | | | | |
| | impurity | | | | |
| | Total | 0.20 | 0.37 | 0.49 | 0.26 |
| | impurities | | | | |

Note:
(2) impurities A, B, and C are known specific impurities according to the quality standards of reference formulations, wherein impurity A is RRT0.78, impurity B is RRT1.2, and impurity C is RRT2.0.

As shown in Table 15, in Comparative Example I, the combination of lactic acid/sodium hydroxide was used as a buffer, and the impurities of the resultant lyophilized lurbinectedin composition increased from 0.20 to 0.49 after storage at 40° C. for 1 month; the stability of Formulation Example VI of the present disclosure and the stability of Formulation Example VII of the present disclosure were comparable to that of the Reference Example I, the stability of the Formulation Example IX was slightly superior to that of Comparative Example I, and the stability of Formulation Example V and the stability of Formulation Example VIII were significantly superior to that of Comparative Example I. In summary, the stabilization effects of the formulas of the present disclosure are superior to that of Comparative Example I, suggesting that the stability of lurbinectedin in the storage process can be ensured.

### Example 11: Preparation Methods for Different Solutions Before Lyophilization

Taking the preparation of Formulation Example I as an example, three different methods for preparing the solution before lyophilization were designed:

### The First Preparation Method:

see Example 1.

### The Second Preparation Method:

1. 40 mg of lurbinectedin was added to 33.71 mg of phosphoric acid, and the mixture was stirred to completely dissolve the drug, so as to obtain a drug solution;
2. 544 mg of potassium dihydrogen phosphate and 8 g of sucrose were added to 70% of water for injection, and the mixture was stirred until complete dissolution and adjusted to pH 4.0±0.2 by using a phosphoric acid solution or a potassium hydroxide solution;
3. The solution obtained in step 1 was added to the solution obtained in step 2, and the mixture was uniformly stirred and adjusted to pH 4.0±0.2 with a phosphoric acid solution or a potassium hydroxide solution;
4. The formulated solution was adjusted to the total weight and filled into vials before the vials were half-stoppered.

### The Third Preparation Method:

1. 40 mg of lurbinectedin was added to 33.71 mg of phosphoric acid and 544 mg of potassium dihydrogen phosphate, and the mixture was stirred to completely dissolve the drug, so as to obtain a drug solution;
2. 8 g of sucrose was added to 70% of water for injection, and the mixture was stirred until complete dissolution and adjusted to pH 4.0±0.2 by using a phosphoric acid solution or a potassium hydroxide solution;
3. The solution obtained in step 1 was added to the solution obtained in step 2, and the mixture was uniformly stirred and adjusted to pH 4.0±0.2 with a phosphoric acid solution or a potassium hydroxide solution;
4. The formulated solution was adjusted to the total weight and filled into vials before the vials were half-stoppered.

The three solutions were then lyophilized to obtain the lyophilized formulations, and the appearance, water content, and related substances were inspected. The lyophilized formulations were tested for storage stability in an acceleration condition (25° C., 60% RH), and the results are shown in Table 16:

TABLE 16
The stability study of the lyophilized formulation produced by different
preparation methods

| | | Preparation | | Preparation | | Preparation | |
|---|---|---|---|---|---|---|---|
| | | method I | | method II | | method III | |
| Time | | 0 days | 3M | 0 days | 3M | 0 days | 3M |
| Appearance | | White | White | White | White | White | White |
| | | solid | solid | solid | solid | solid | solid |
| | | mass | mass | mass | mass | mass | mass |
| Water content (%) | | 1.99 | 2.20 | 2.12 | 2.31 | 2.05 | 2.22 |
| Related substances | RRT0.16 | / | / | / | / | / | / |
| (area | RRT0.23 | / | / | / | / | / | / |
| normalization, %) | RRT0.41 | / | / | / | / | / | / |
| | RRT0.61 | / | / | / | / | / | / |
| | RRT0.69 | / | / | / | / | / | / |
| | RRT0.84 | 0.11 | 0.16 | 0.10 | 0.17 | 0.12 | 0.16 |
| | RRT1.10 | / | / | / | / | / | / |
| | RRT1.28 | 0.06 | 0.06 | 0.05 | 0.07 | 0.06 | 0.07 |
| | RRT1.36 | 0.06 | 0.07 | 0.07 | 0.07 | 0.07 | 0.07 |
| | Maximum unknown | 0.11 | 0.16 | 0.12 | 0.18 | 0.11 | 0.19 |
| | individual impurity | | | | | | |
| | Total impurities | 0.39 | 0.41 | 0.41 | 0.44 | 0.40 | 0.42 |

In addition, the appearance of the lyophilized formulations was also inspected, and the results are shown in Table 17:

TABLE 17
Appearance of the lyophilized formulation produced by different
preparation methods

| | |
|---|---|
| Preparation method | Appearance |
| Preparation method I | No defects in appearance |
| Preparation method II | Slight shrink at bottom |
| Preparation method III | Slight shrink at bottom |

It can be seen from the contents in Tables 16 and 17 that the formulations prepared by the three preparation methods exhibited no significant changes in appearance and related substances after storage in the acceleration condition for 3 months, and no significant differences were seen among the groups. Among these, preparation method I exhibited better water content and appearance than those of the other two groups, and was thus used as the preferred preparation method for the solution before lyophilization to ensure higher product quality.

### Example 12: Different Lyophilization Procedures

A solution before lyophilization was prepared according to the formulation and preparation method in Example 1 and lyophilized according to the following three lyophilization processes.

TABLE 18
Lyophilization Process I

| | | | Time for | |
|---|---|---|---|---|
| | | | temperature/ | |
| | | | pressure | |
| Stage | Temperature | Vacuum | rise and fall | Duration |
| Low-temperature | 5° C. | N/A | N/A | N/A |
| feeding | | | | |
| Pre-freezing | 5° C. | N/A | N/A | 30 min |
| | −5° C. | N/A | 00:10 | 50 min |
| | −40° C. | N/A | 01:30 | 4 h |
| Primary drying | −40° C. | 0.10 mbar | 00:20 | N/A |
| | −25° C. | 0.10 mbar | 02:00 | 50 h |
| Secondary drying | −25° C. | 0.03 mbar | 00:10 | N/A |
| | 25° C. | 0.03 mbar | 02:00 | 28 h |
| Stoppering | 25° C. | 600 mbar | N/A | N/A |

TABLE 19
Lyophilization Process II

| | | | Time for | |
|---|---|---|---|---|
| | | | temperature/ | |
| | | | pressure | |
| Stage | Temperature | Vacuum | rise and fall | Duration |
| Low-temperature | 5° C. | N/A | N/A | N/A |
| feeding | | | | |
| Pre-freezing | 5° C. | N/A | N/A | 30 min |
| | −5° C. | N/A | 00:10 | 50 min |
| | −40° C. | N/A | 01:30 | 8 h |
| Primary drying | −40° C. | 0.10 mbar | 00:20 | N/A |
| | −25° C. | 0.10 mbar | 02:00 | 36 h |
| Secondary drying | −25° C. | 0.10 mbar | 00:10 | N/A |
| | 25° C. | 0.10 mbar | 02:00 | 28 h |
| Stoppering | 25° C. | 600 mbar | N/A | N/A |

TABLE 20
Lyophilization Process III

| | | | Time for | |
|---|---|---|---|---|
| | | | temperature/ | |
| | | | pressure | |
| Stage | Temperature | Vacuum | rise and fall | Duration |
| Low-temperature | 5° C. | N/A | N/A | N/A |
| feeding | | | | |
| Pre-freezing | 5° C. | N/A | N/A | 30 min |
| | −5° C. | N/A | 00:10 | 50 min |
| | −40° C. | N/A | 01:30 | 4 h |
| Primary drying | −40° C. | 0.10 mbar | 00:20 | N/A |
| | −25° C. | 0.10 mbar | 02:00 | 50 h |
| Secondary drying | −25° C. | 0.10 mbar | 00:10 | N/A |
| | 25° C. | 0.10 mbar | 02:00 | 28 h |
| Stoppering | 25° C. | 600 mbar | N/A | N/A |

The appearance, water content, and related substances were inspected. The lyophilized formulations were tested for storage stability in an acceleration condition (25° C., 60% RH), and the results are shown in Table 21:

TABLE 21
The stability characteristics of the lyophilized composition produced by different
lyophilization processes

| | | Lyophilization | | Lyophilization | | Lyophilization | |
|---|---|---|---|---|---|---|---|
| | | Process I | | Process II | | Process III | |
| Time | | 0 days | 3M | 0 days | 3M | 0 days | 3M |
| Appearance | | White | White | White | White | White | White |
| | | solid | solid | solid | solid | solid | solid |
| | | mass | mass | mass | mass | mass | mass |
| Water content (%) | | 1.99 | 2.20 | 2.72 | 2.94 | 2.36 | 2.57 |
| Related | RRT0.16 | / | / | / | / | / | / |
| substances | RRT0.23 | / | / | / | / | / | / |
| (area | RRT0.41 | / | / | / | / | / | / |
| normalization, %) | RRT0.61 | / | / | / | / | / | / |
| | RRT0.69 | / | / | / | / | / | / |
| | RRT0.84 | 0.11 | 0.16 | 0.12 | 0.17 | 0.11 | 0.17 |
| | RRT1.10 | / | / | / | / | / | / |
| | RRT1.28 | 0.06 | 0.06 | 0.05 | 0.06 | 0.06 | 0.07 |
| | RRT1.36 | 0.06 | 0.07 | 0.06 | 0.06 | 0.07 | 0.06 |
| | Maximum unknown | 0.11 | 0.16 | 0.12 | 0.17 | 0.12 | 0.19 |
| | individual impurity | | | | | | |
| | Total impurities | 0.39 | 0.41 | 0.42 | 0.45 | 0.44 | 0.49 |

In addition, the appearance of the lyophilized formulations was also inspected, and the results are shown in Table 22:

TABLE 22
Appearance of the lyophilized formulation produced by different
lyophilization processes

| | |
|---|---|
| Preparation method | Appearance |
| Lyophilization Process I | No defects in appearance |
| Lyophilization Process II | Slight shrink at bottom |
| Lyophilization Process III | Slight shrink at bottom |

It can be seen from the contents in Tables 21 and 22 that the formulations prepared by the three lyophilization processes exhibited no significant changes in appearance and related substances after storage in the acceleration condition for 3 months, and no significant differences were seen among the groups. Among these, Lyophilization Process I exhibited better water content and appearance than those of the other two groups, and was thus selected as the preferred lyophilization process.

### Example 13: Different Lyophilization Processes

A solution before lyophilization was prepared according to the formula and preparation method in Example 5 and lyophilized according to the Lyophilization Process IV in Table 23:

TABLE 23
Lyophilization Process IV

| | | | Time for | |
|---|---|---|---|---|
| | | | temperature/ | |
| | | | pressure | |
| Stage | Temperature | Vacuum | rise and fall | Duration |
| Low-temperature | 5° C. | N/A | N/A | N/A |
| feeding | | | | |
| Pre-freezing | −5° C. | N/A | 10 min | 1 h |
| | −45° C. | N/A | 30 min | 6 h |
| Primary drying | −25° C. | 0.10 mbar | 60 min | 60~80 h |
| | −10° C. | 0.10 mbar | 100 min | 10~20 h |
| Secondary drying | 40° C. | 0.10 mbar | 330 min | 30 h |
| | 40° C. | N/A | 1 min | 4~16 h |

The appearance, water content, and related substances of the lyophilized formulation prepared by Lyophilization Process IV were inspected, and the results are shown in Table 24.

TABLE 24
The stability study of the lyophilized formulation produced by Lyophilization
Process IV

| | | | | | | | Light | | |
|---|---|---|---|---|---|---|---|---|---|
| | | | High temperature | | High temperature | | exposure | | |
| Inspection | Limit | | (40° C.) | | (60° C.) | | 1.2 × 10⁶ | High humidity | |
| item | requirement | 0 days | 10 days | 30 days | 10 days | 30 days | lx · h | 10 days | 30 days |
| Appearance | White or | White | White | White | White | Pale | White | White | White |
| | off-white | solid | solid | solid | solid | yellow | solid | solid | solid |
| | solid mass | mass | mass | mass | mass | solid | mass | mass | mass |
| | or powder | | | | | mass | | | |
| Reconstitution | No more | 11.93 | 11.67 | 10.47 | 12.47 | 11.81 | 10.86 | 13.60 | 11.21 |
| time | than 3 min | | | | | | | | |
| (s) | | | | | | | | | |
| Acidity | pH 3.5 to | 4.0 | 4.0 | 4.1 | 4.0 | 4.1 | 4.0 | 4.0 | 4.1 |
| | 4.5 | | | | | | | | |
| Solution | Clear and | Clear | Clear | Clear | Clear | Clear | Clear | Clear | Clear |
| clarity | colorless; if | and | and | and | and | and | and | and | and |
| and | colored, | colorless | colorless | colorless | colorless | colorless | colorless | colorless | colorless |
| color | should not | | | | | | | | |
| | be deeper | | | | | | | | |
| | than | | | | | | | | |
| | standard | | | | | | | | |
| | colorimetric | | | | | | | | |
| | solution | | | | | | | | |
| | yellow 2. | | | | | | | | |
| Related | Impurity | 0.04 | 0.05 | 0.06 | 0.15 | 0.58 | 0.04 | 0.04 | 0.04 |
| | A ≤ 0.8 | | | | | | | | |
| substances | Impurity | / | 0.02 | 0.02 | 0.03 | 0.02 | 0.03 | / | / |
| | B ≤ 0.3 | | | | | | | | |
| (%) | Impurity | 0.06 | 0.06 | 0.06 | 0.06 | 0.05 | 0.06 | 0.06 | 0.06 |
| | C ≤ 0.3 | | | | | | | | |
| | Other | 0.22 | 0.26 | 0.33 | 0.53 | 0.67 | 0.19 | 0.23 | 0.24 |
| | individual | | | | | | | | |
| | impurity ≤ | | | | | | | | |
| | 0.3 | | | | | | | | |
| | Total | 0.41 | 0.55 | 0.74 | 1.59 | 3.23 | 0.46 | 0.46 | 0.49 |
| | impurities ≤ | | | | | | | | |
| | 1.3 | | | | | | | | |
| Water content | ≤3.0 | 0.60 | 0.80 | 0.62 | 0.82 | 0.84 | 0.74 | 0.60 | 0.66 |
| (%) | | | | | | | | | |

Note:
(1) “/” indicates not detected;
(2) impurities A, B, and C are known specific impurities according to the quality standards of reference formulations, wherein impurity A is RRT0.78, impurity B is RRT1.2, and impurity C is RRT2.0.

As can be seen from Table 24, when the lyophilized formulation prepared by Lyophilization Process IV was let to stand in acceleration conditions (40° C. or 60° C.) for 10 or 30 days, the lyophilized formulation exhibited no significant changes in appearance and related substances and a great reduction in water content, and is thus a lyophilized formulation with higher quality.

Various embodiments of the present disclosure have been described above, and the above description is illustrative, rather than exhaustive, and is not limited to the disclosed embodiments. Many modifications and variations will be apparent to those of ordinary skill in the art without departing from the scope and spirit of the described embodiments. The terms used herein are selected to best explain the principles of the various embodiments, practical application, or technical improvements to the prior art, or to enable others of ordinary skill in the art to understand the various embodiments disclosed herein.

1. A pharmaceutical composition comprising lurbinectedin, wherein the pharmaceutical composition is a lyophilized composition comprising: lurbinectedin, a buffer derived from an inorganic acid, and a bulking agent.

2. The pharmaceutical composition according to claim 1, wherein the buffer derived from the inorganic acid is a combination of the inorganic acid and a salt of the inorganic acid or a combination of the inorganic acid and a base.

3. The pharmaceutical composition according to claim 2, wherein the combination of the inorganic acid and the salt of the inorganic acid is a combination of phosphoric acid and a phosphate salt; the combination of the inorganic acid and the base is a combination of hydrochloric acid and the base, a combination of sulfuric acid and the base, or a combination of phosphoric acid and the base.

4. The pharmaceutical composition according to claim 3, wherein the base is any one of sodium carbonate, potassium carbonate, NH₄OH, lithium hydroxide, sodium hydroxide, potassium hydroxide, sodium bicarbonate, and potassium bicarbonate.

5. The pharmaceutical composition according to claim 1, wherein the bulking agent is a disaccharide selected from any one of sucrose, trehalose, and lactose.

6. The pharmaceutical composition according to claim 1, wherein in the solution prior to lyophilization of the lyophilized composition, the concentration of lurbinectedin is 0.4-0.6 mg/mL.

7. The pharmaceutical composition according to claim 1, wherein the molar ratio of lurbinectedin to the bulking agent is 1:440-1:480.

8. The pharmaceutical composition according to claim 1, wherein the pH of the solution prior to lyophilization of the lyophilized composition is 3 to 5.

9. The pharmaceutical composition according to claim 1, wherein the pharmaceutical composition consists of lurbinectedin, phosphoric acid, potassium dihydrogen phosphate, sucrose, potassium hydroxide, and water; the concentration of the lurbinectedin is 0.4 to 0.6 mg/mL, and the pH of the solution prior to lyophilization of the lyophilized composition is 3 to 5.

10. The pharmaceutical composition according to claim 1, wherein the pharmaceutical composition consists of lurbinectedin, phosphoric acid, sodium dihydrogen phosphate, sucrose, and water; the concentration of lurbinectedin is 0.4 to 0.6 mg/mL, and the pH of the solution prior to lyophilization of the lyophilized composition is 3 to 5.

11. The pharmaceutical composition according to claim 9, wherein each 80 mL of the pharmaceutical composition comprises the following components

| component | amount |
|---|---|
| lurbinectedin | 40 mg |
| sucrose | 8 g |
| phosphoric acid | 33.71 mg |
| potassium dihydrogen phosphate | 544 mg |
| potassium hydroxide/phosphoric acid | proper amount |
| water for injection | the balance to 80 mL |

12. The pharmaceutical composition according to claim 10, wherein each 80 mL of the pharmaceutical composition comprises the following components:

| component | amount |
|---|---|
| lurbinectedin | 40 mg |
| sucrose | 8 g |
| phosphoric acid | 11.5 mg |
| sodium dihydrogen phosphate | 294 mg |
| water for injection | the balance to 80 mL |

13. A method for preparing the pharmaceutical composition according to claim 1, comprising the following steps: mixing lurbinectedin, a buffer derived from an inorganic acid, a bulking agent, and water to form a mixture; and adjusting the pH of the mixture to obtain the pharmaceutical composition.

14. The method according to claim 13, wherein the buffer derived from the inorganic acid is a combination of the inorganic acid and a salt of the inorganic acid or a combination of the inorganic acid and a base, and the method is any one of the following methods:
preparation method I:
1) mixing lurbinectedin with an inorganic acid;
2) adding water, a base/a salt of the inorganic acid; and
3) adding a bulking agent, and adjusting the pH to obtain the pharmaceutical composition;
preparation method II:
1) mixing lurbinectedin with an inorganic acid;
2) mixing a base/a salt of the inorganic acid, a bulking agent, and water, and adjusting the pH; and
3) mixing the solution obtained in step 1) and the solution obtained in step 2), and adjusting the pH to obtain the pharmaceutical composition; or
preparation method III:
1) mixing an inorganic acid, a salt of the inorganic acid, and lurbinectedin;
2) mixing a bulking agent with water, and adjusting the pH; and
3) mixing the solution obtained in step 1) and the solution obtained in step 2), and adjusting the pH to obtain the pharmaceutical composition.

15. The method according to claim 13, further comprising a lyophilization process after adjusting the pH, wherein the lyophilization process comprises three stages: a pre-freezing, a primary drying, and a secondary drying; for the pre-freezing, the temperature is −50 to 5° C.; for the primary drying, the temperature is −40 to −10° C., wherein the vacuum pressure is maintained at 0.1 to 0.5 mbar; for the secondary drying, the temperature is −25 to 25° C., wherein the vacuum pressure is maintained at 0.01 to 0.5 mbar or 0.05 to 0.5 mbar.

16. The method according to claim 13, further comprising a lyophilization process after adjusting the pH, wherein the lyophilization process comprises three stages: a pre-freezing, a primary drying, and a secondary drying; for the pre-freezing, the temperature is −50 to 5° C.; for the primary drying, the temperature is −40 to −10° C., wherein the vacuum pressure is maintained at 0.05 to 0.5 mbar; for the secondary drying, the temperature is −25 to 45° C., wherein the vacuum pressure is maintained at 0.01 to 0.5 mbar or 0.05 to 0.5 mbar.

17. A method for treating tumors comprising administering to a subject in need thereof the pharmaceutical composition according to claim 1.

18. A method for preparing the pharmaceutical composition according to claim 11, comprising the following steps: mixing lurbinectedin, a buffer derived from an inorganic acid, a bulking agent, and water to form a mixture; and adjusting pH of the mixture to obtain the pharmaceutical composition.

19. A method for preparing the pharmaceutical composition according to claim 12, comprising the following steps: mixing lurbinectedin, a buffer derived from an inorganic acid, a bulking agent, and water to form a mixture; and adjusting pH of the mixture to obtain the pharmaceutical composition.

20. A method for treating tumors comprising administering to a subject in need thereof the pharmaceutical composition according to claim 12.
